(19) United States
(12) Patent Application Publication
(10) Pub. No.: US 2020/0077983 A1
(43) Pub. Date: Mar. 12, 2020

(54) ULTRASONIC DIAGNOSTIC APPARATUS, MEDICAL IMAGE PROCESSING APPARATUS, AND NON-TRANSITORY COMPUTER MEDIUM STORING COMPUTER PROGRAM

(71) Applicant: CANON MEDICAL SYSTEMS CORPORATION, Otawara-shi (JP)

(72) Inventors: Asuka Ozawa, Nasushiobara (JP); Hiroki Yoshiara, Kawasaki (JP); Takeshi Sugio, Otawara (JP); Tomio Nabatame, Otawara (JP); Takashi Masuda, Utsunomiya (JP)

(73) Assignee: CANON MEDICAL SYSTEMS CORPORATION, Otawara-shi (JP)

(21) Appl. No.: 16/568,514

(22) Filed: Sep. 12, 2019

(30) Foreign Application Priority Data
Sep. 12, 2018 (JP) 2018-170793

(51) Int. Cl. A61B 8/08; A61B 8/06

(52) U.S. Cl. CPC A61B 8/481; G01S 7/52041; A61B 8/488; A61B 8/06

(57) ABSTRACT

The ultrasonic diagnostic apparatus according to a present embodiment includes processing circuitry. The processing circuitry is configured to acquire a contrast image and a vascular structure image which includes the vascular structure in an image acquisition mode capable of capturing the vascular structure. The processing circuitry is configured to extract data indicating the vascular structure from the vascular structure image. The processing circuitry is configured to superimpose the data indicating the vascular structure on the contrast image to generate a superimposed image. The processing circuitry is configured to display the superimposed image on a display.

### CROSS-REFERENCE TO RELATED APPLICATION

This application is based upon and claims the benefit of priority from Japanese Patent Application No. 2018-170793, filed on Sep. 12, 2018, the entire contents of each of which are incorporated herein by reference.

### FIELD

An embodiment as an aspect of the present invention relates to an ultrasonic diagnostic apparatus, a medical image processing apparatus and a non-transitory computer readable medium storing a computer program.

### BACKGROUND

In the medical field, an ultrasonic diagnostic apparatus is used for imaging the inside of a subject using ultrasonic waves generated by multiple transducers (piezoelectric transducers) of an ultrasonic probe. The ultrasonic diagnostic apparatus causes the ultrasonic probe, which is connected to the ultrasonic diagnostic apparatus, to transmit ultrasonic waves into the subject, generates an echo signal based on reflected waves, and acquires a desired ultrasonic image by image processing.

According to a two-screen display of vascular data and a tissue image in the ultrasonic diagnostic apparatus, it is possible for an operator to observe a vascular structure while viewing a scan position based on the tissue image. However, a contrast image cannot be observed simultaneously. On the other hand, according to a two-screen display of the contrast image and the tissue image in the ultrasonic diagnostic apparatus, it is possible for the operator to observe the contrast image while viewing a scan position based on the tissue image. However, in the late phase, since a contrast agent may spread from the blood vessel to tissues, it may be difficult to grasp the vascular structure in the two-screen display.

### BRIEF DESCRIPTION OF THE DRAWINGS

FIG. 1 is a schematic diagram showing a configuration of an ultrasonic diagnostic apparatus according to a present embodiment.

FIG. 2 is a block diagram showing functions of the ultrasonic diagnostic apparatus according to the present embodiment.

FIG. 3 is a flowchart showing the operation of the ultrasonic diagnostic apparatus according to the present embodiment.

Each of FIGS. 4A and 4B is a diagram showing an example of a CHI image in the ultrasonic diagnostic apparatus according to the present embodiment.

FIG. 5 is a diagram showing an example of a vascular structure image showing a microvascular structure in the ultrasonic diagnostic apparatus according to the present embodiment.

Each of FIGS. 6A and 6B is a diagram showing a first example of a superimposed image in the ultrasonic diagnostic apparatus according to the present embodiment.

Each of FIGS. 7A and 7B is a diagram showing a superimposed image according to a comparative example.

Each of FIGS. 8A and 8B is a diagram showing a second example of the superimposed image in the ultrasonic diagnostic apparatus according to the present embodiment.

Each of FIGS. 9A and 9B is a diagram showing an example of a display image in the ultrasonic diagnostic apparatus according to the present embodiment.

Each of FIGS. 10A and 10B is a diagram for explaining a superimposed image (three-dimensional image) in the ultrasonic diagnostic apparatus according to the present embodiment.

FIG. 11 is a schematic diagram showing a configuration of a medical image processing apparatus according to a present embodiment.

FIG. 12 is a block diagram showing functions of the medical image processing apparatus according to the present embodiment.

FIG. 13 is a diagram for explaining a concept of switching the display of the superimposed image to a parallel display of the CHI image, the vascular structure image and the tissue image as elements thereof in the medical image processing apparatus according to the present embodiment.

### DETAILED DESCRIPTION

An ultrasonic diagnostic apparatus, a medical image processing apparatus and a non-transitory computer readable medium storing a computer program according to a present embodiment will be described with reference to the accompanying drawings.

The ultrasonic diagnostic apparatus according to a present embodiment includes processing circuitry. The processing circuitry is configured to acquire a contrast image and a vascular structure image which includes the vascular structure in an image acquisition mode capable of capturing the vascular structure. The processing circuitry is configured to extract data indicating the vascular structure from the vascular structure image. The processing circuitry is configured to superimpose the data indicating the vascular structure on the contrast image to generate a superimposed image. The processing circuitry is configured to display the superimposed image on a display.

1. Ultrasonic Diagnostic Apparatus

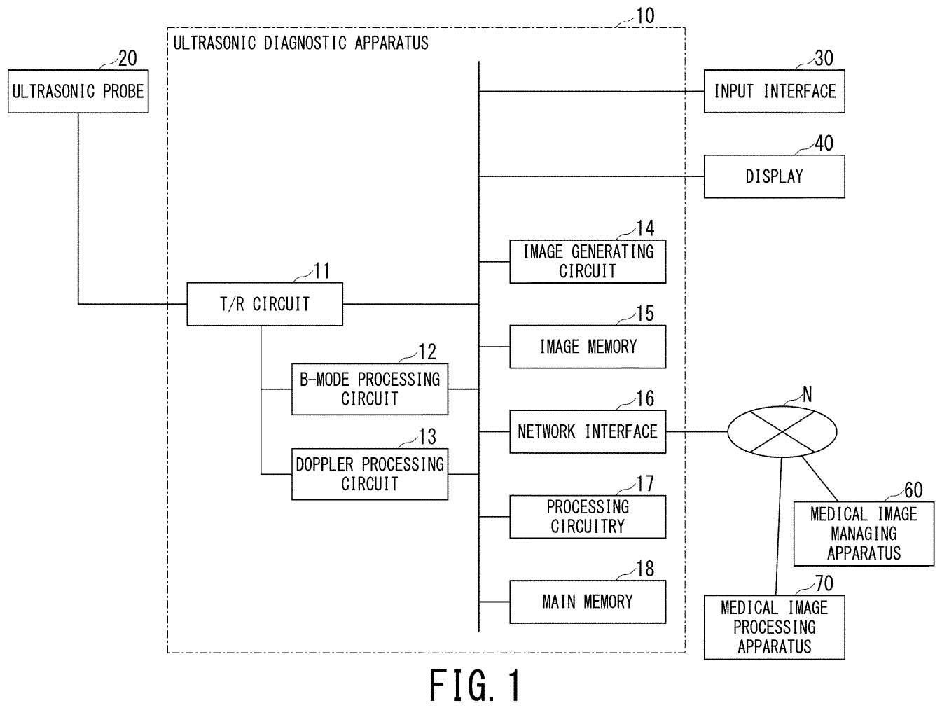

FIG. 1 is a schematic diagram showing a configuration of an ultrasonic diagnostic apparatus according to a present embodiment.

FIG. 1 shows an ultrasonic diagnostic apparatus 10 according to a present embodiment. FIG. 1 shows an ultrasonic probe 20, an input interface 30 and a display 40.

It should be noted that a device in which at least one of the ultrasonic probe 20, the input interface 30, and the display 40 is added to the ultrasonic diagnostic apparatus 10 may be referred to as an ultrasonic diagnostic apparatus in some cases. In the following description, a case where all of the ultrasonic probe 20, the input interface 30, and the display 40 are provided outside the ultrasonic diagnostic apparatus 10 will be described.

The ultrasonic diagnostic apparatus 10 includes a transmitting and receiving (T/R) circuit 11, a B-mode processing circuit 12, a Doppler processing circuit 13, an image generating circuit 14, an image memory 15, a network interface 16, processing circuitry 17 and a main memory 18. The circuits 11 to 14 are configured by an application specific integrated circuit (ASIC) or the like. However, the present invention is not limited to this case, and all or a part of the functions of the circuits 11 to 14 may be realized by the processing circuitry 17 executing a program.

The T/R circuit 11 has a transmitting circuit and a receiving circuit (not shown). Under the control of the processing circuitry 17, the T/R circuit 11 controls transmission directivity and reception directivity in transmission and reception of ultrasonic waves. The case where the T/R circuit 11 is provided in the ultrasonic diagnostic apparatus 10 will be described, but the T/R circuit 11 may be provided in the ultrasonic probe 20, or may be provided in both of the ultrasonic diagnostic apparatus 10 and the ultrasonic probe 20. The T/R circuit 11 is one example of a transmitter-and-receiver.

The transmitting circuit has a pulse generating circuit, a transmission delay circuit, a pulsar circuit and the like, and supplies a drive signal to ultrasonic transducers. The pulse generating circuit repeatedly generates rate pulses for forming transmission ultrasonic waves at a predetermined rate frequency. The transmission delay circuit converges the ultrasonic waves generated from the ultrasonic transducer of the ultrasonic probe 20 into a beam shape, and gives a delay time for each piezoelectric transducer necessary for determining the transmission directivity to each rate pulse generated by the pulse generating circuit. In addition, the pulsar circuit applies drive pulses to each ultrasonic transducer at a timing based on the rate pulses. The transmission delay circuit arbitrarily adjusts the transmission direction of the ultrasonic beam transmitted from a piezoelectric transducer surface by changing the delay time given to each rate pulse.

The receiving circuit has an amplifier circuit, an A/D (Analog to Digital) converter, an adder, and the like, and receives the echo signal received by the ultrasonic transducers and performs various processes on the echo signal to generate echo data. The amplifier circuit amplifies the echo signal for each channel, and performs gain correction processing. The A/D converter A/D-converts the gain-corrected echo signal, and gives a delay time necessary for determining the reception directivity to the digital data. The adder adds the echo signal processed by the A/D converter to generate echo data. By the addition processing of the adder, the reflection component from the direction corresponding to the reception directivity of the echo signal is emphasized.

Under the control of the processing circuitry 17, the B-mode processing circuit 12 receives the echo data from the receiving circuit, performs logarithmic amplification, envelope detection processing and the like, thereby generating data (two-dimensional (2D) or three-dimensional (3D) data) whose signal intensity is represented by brightness of luminance. This data is generally called “B-mode data”. The B-mode processing circuit 12 is one example of a B-mode processer.

The B-mode processing circuit 12 may change the frequency band to be visualized by changing the detection frequency by filter processing. By using the filtering function of the B-mode processing circuit 12, harmonic imaging such as the contrast harmonic imaging (CHI) or the tissue harmonic imaging (THI) is performed. That is, the B-mode processing circuit 12 may separate the reflected waves from within a subject into which the contrast agent is injected into harmonic data (or sub-frequency data) and fundamental wave data. The harmonic data (or sub-frequency data) corresponds to reflected waves with a harmonic component whose reflection source is the contrast agent (microbubbles or bubbles) in the subject. The fundamental wave data corresponds to reflected waves with a fundamental wave component whose reflection source is tissue in the subject. The B-mode processing circuit 12 generates B-mode data for generating contrast image data based on the reflected wave data (received signal) of the harmonic component, and generates B-mode data for generating fundamental wave image data based on the reflected wave data (received signal) with the fundamental wave component.

In the THI by using the filter processing function of the B-mode processing circuit 12, it is possible to separate harmonic data or sub-frequency data which is reflected wave data (received signal) of a harmonic component from reflected wave data of the subject. Then, the B-mode processing circuit 12 generates B-mode data for generating tissue image data in which the noise component is removed from the reflected wave data (received signal) of the harmonic component.

When the CHI or THI harmonic imaging is performed, the B-mode processing circuit 12 may extract the harmonic component by a method different from the method using the above-described filtering. In harmonic imaging, an imaging method called the amplitude modulation (AM) method, the phase modulation (PM) method or the AM-PM method in which the AM method and the PM method are combined is performed. In the AM method, the PM method, and the AM-PM method, ultrasonic transmission with different amplitudes and phases is performed multiple times on the same scanning line. Thereby, the T/R circuit 11 generates and outputs multiple reflected wave data (received signals) in each scanning line. The B-mode processing circuit 12 extracts harmonic components by performing addition/subtraction processing according to the modulation method on the multiple reflected wave data (received signals) of each scanning line. The B-mode processing circuit 12 performs envelope detection processing etc. on the reflected wave data (received signal) of the harmonic component to generate B-mode data.

For example, when the PM method is performed, the T/R circuit 11 controls the ultrasonic waves of the same amplitude and of reversed-phase polarities each other, for example (−1, 1), to be transmitted twice by each scanning line under a scan sequence set by the processing circuit 17. The T/R circuit 11 generates a reception signal based on transmission of “−1” and a reception signal based on transmission of “1”. The B-mode processing circuit 12 adds these two reception signals. As a result, the fundamental wave component is removed, and a signal in which the second harmonic component mainly remains is generated. Then, the B-mode processing circuit 12 performs envelope detection processing and the like on this signal to generate B-mode data using THI or CHI.

Alternatively, for example, in the THI, an imaging method using the second harmonic component and a difference tone component included in the received signal has been put to practical use. In the imaging method using the difference tone component, transmission ultrasonic waves are transmitted from the ultrasonic probe 20, the transmission ultrasonic waves having, for example, a composite waveform in which a first fundamental waves with a center frequency “f1” and a second fundamental waves with a center frequency “f2” larger than the center frequency “f1” are combined. Such a composite waveform is a waveform in which a waveform with the first fundamental waves and a waveform with the second fundamental waves whose phases are adjusted with each other are combined such that the difference tone component with the same polarity as the second harmonic component is generated. The T/R circuit 11 transmits the transmission ultrasonic waves of the composite waveform, for example, twice while inverting the phase. In such a case, for example, the B-mode processing circuit 12 removes the fundamental wave component by adding two received signals, and performs an envelope detection process etc. after extracting a harmonic component in which the difference tone component and the second harmonic component are mainly left.

Under the control of the processing circuitry 17, the Doppler processing circuit 13 frequency-analyzes the phase information from the echo data from the receiving circuit, thereby generating data (two-dimensional or three-dimensional data) acquired by extracting moving data of moving subject such as average speed, dispersion, power and the like for multiple points. This data is generally called “Doppler data”. In the present embodiment, the moving subject is, for example, blood flow, tissue such as heart wall, or contrast agent. The Doppler processing circuit 13 is one example of a Doppler processer.

Under the control of the processing circuitry 17, the image generating circuit 14 generates an ultrasonic image expressed in a predetermined luminance range as image data based on the echo signal received by the ultrasonic probe 20. For example, the image generating circuit 14 generates a B-mode image in which the intensity of the reflected waves is expressed in luminance from the two-dimensional B-mode data generated by the B-mode processing circuit 12 as the ultrasonic image. Further, the image generating circuit 14 generates, as the ultrasonic image, a color Doppler image representing moving state information from the two-dimensional Doppler data generated by the Doppler processing circuit 13 such as an average velocity image, a dispersed image, a power image, or a combined image thereof. The image generating circuit 14 is one example of an image generator.

In the present embodiment, the image generating circuit 14 generally converts (scan converts) a scanning line of ultrasonic scanning into a scanning line of a video format represented by a television or the like, and generates ultrasonic image data for display. Specifically, the image generating circuit 14 performs coordinate conversion in accordance with the scanning form of the ultrasonic waves by the ultrasonic probe 20, thereby generates the ultrasonic image data for display. In addition to the scan conversion, the image generating circuit 14 may perform various image processing such as smoothing processing for regenerating an average luminance image using image frames after scan conversion, and edge enhancement processing using a differential filter in the image. Further, the image generating circuit 14 superimposes character information, scales, body marks, and the like of various parameters on the ultrasonic image data.

That is, both the B-mode data and the Doppler data are ultrasonic image data before the scan conversion processing. The data generated by the image generating circuit 14 is ultrasonic image data for display after the scan conversion processing. The B-mode data and the Doppler data are also called raw data. The image generating circuit 14 generates two-dimensional ultrasonic image data for display from the two-dimensional ultrasonic image data before the scan conversion processing.

The image generating circuit 14 generates three-dimensional B-mode image data by performing coordinate conversion on the three-dimensional B-mode data generated by the B-mode processing circuit 12. The image generating circuit 14 generates three-dimensional Doppler image data by performing coordinate conversion on the three-dimensional

Doppler data generated by the Doppler processing circuit 13. The image generating circuit 14 generates “3D B-mode image data or 3D Doppler image data” as “3D ultrasonic image data (volume data)”.

The image generating circuit 14 performs a rendering processing on the volume data in order to generate various two-dimensional image data for displaying the volume data on the display 40. As the rendering processing, the image generating circuit 14 performs, for example, multi planer reconstruction (MPR) processing based on the volume data, thereby generates MPR image data. As the rendering processing, the image generating circuit 14 performs, for example, a volume rendering (VR) processing that generates two-dimensional image data reflecting three-dimensional information.

The image memory 15 includes memory cells in two axial directions per frame, and includes a two-dimensional memory which is a memory having the memory cells for frames. Under the control of the processing circuitry 17, the two-dimensional memory as the image memory 15 stores the ultrasonic image of one frame or the ultrasonic images frames generated by the image generating circuit 14 as two-dimensional image data. The image memory 15 is one example of a storage.

Under the control of the processing circuitry 17, the image generating circuit 14 performs three-dimensional reconstruction on the ultrasonic image arranged in the two-dimensional memory as the image memory 15 by interpolation processing, if necessary, thereby generating an ultrasonic image as volume data in a three-dimensional memory as the image memory 15. With respect to an interpolation processing method, a known technique is used.

The image memory 15 may include a three-dimensional memory which is a memory having memory cells in three axial directions (X-axis, Y-axis, and Z-axis direction). The three-dimensional memory as the image memory 15 stores the ultrasonic image generated by the image generating circuit 14 as volume data under the control of the processing circuitry 17.

The network interface 16 implements various information communication protocols according to the network form. The network interface 16 connects the ultrasonic diagnostic apparatus 10 and other devices such as the external medical image managing apparatus 60 and the medical image processing apparatus 70 according to these various protocols. An electrical connection or the like via an electronic network is applied to this connection. In the present embodiment, the electronic network means an entire information communication network using telecommunications technology. The electronic network includes a wired/wireless hospital backbone local area network (LAN) and the Internet network, as well as a telephone communication line network, an optical fiber communication network, a cable communication network, a satellite communication network, or the like.

Further, the network interface 16 may implement various protocols for non-contact wireless communication. In this case, the ultrasonic diagnostic apparatus 10 can directly transmit/receive data to/from the ultrasonic probe 20, for example, without going through the network. The network interface 16 is one example of a network connector.

The processing circuitry 17 means an ASIC, a programmable logic device, etc. in addition to a dedicated or general purpose central processing unit (CPU), a micro processor unit (MPU), or graphics processing unit (GPU). The programmable logic device may refer to, for example, a simple programmable logic device (SPLD), a complex programmable logic device (CPLD), a field programmable gate array (FPGA).

Further, the processing circuitry 17 may be constituted by a single circuit or a combination of independent circuit elements. In the latter case, the main memory 18 may be provided individually for each circuit element, or a single main memory 18 may store programs corresponding to the functions of the circuit elements. The processing circuitry 17 is one example of a processor.

The main memory 18 is constituted by a semiconductor memory element such as a random access memory (RAM), a flash memory, a hard disk, an optical disk, or the like. The main memory 18 may be constituted by a portable medium such as a universal serial bus (USB) memory and a digital video disk (DVD). The main memory 18 stores various processing programs (including an OS (operating system) and the like besides the application program) used in the processing circuitry 17 and data necessary for executing the programs. In addition, the OS may include a graphical user interface (GUI) which allows the operator to frequently use graphics to display information on the display 40 to the operator and can perform basic operations by the input interface 30. The main memory 18 is one example of a storage.

The ultrasonic probe 20 includes microscopic transducers (piezoelectric elements) on the front surface portion, and transmits and receives ultrasonic waves to a region including a scan target, for example, a region including a lumen. Each transducer is an electroacoustic transducer, and has a function of converting electric pulses into ultrasonic pulses at the time of transmission and converting reflected waves to electric signals (reception signals) at the time of reception. The ultrasonic probe 20 is configured to be small and lightweight, and is connected to the ultrasonic diagnostic apparatus 10 via a cable (or wireless communication).

The ultrasonic probe 20 is classified into types such as a linear type, a convex type, a sector type, etc. depending on a difference in scanning system. The ultrasonic probe 20 is classified into a 1D array probe in which transducers are arrayed in a one-dimensional (1D) manner in the azimuth direction, and a 2D array probe in which transducers are arrayed in two dimensions (2D) manner in the azimuth direction and in the elevation direction, depending on the array arrangement dimension. The 1D array probe includes a probe in which a small number of transducers are arranged in the elevation direction.

In this embodiment, when a 3D scan, that is, a volume scan is executed, the 2D array probe having a scan type such as the linear type, the convex type, the sector type, or the like is used as the ultrasonic probe 20. Alternatively, when the volume scan is executed, the 1D probe having a scan type such as the linear type, the convex type, the sector type and the like and having a mechanism that mechanically oscillates in the elevation direction is used as the ultrasonic probe 20. The latter probe is also called a mechanical 4D probe.

The input interface 30 includes an input device operable by an operator, and a circuit for inputting a signal from the input device. The input device may be a trackball, a switch, a mouse, a keyboard, a touch pad for performing an input operation by touching an operation surface, a touch screen in which a display screen and a touch pad are integrated, a non-contact input circuit using an optical sensor, an audio input circuit, and the like. When the input device is operated by the operator, the input interface 30 generates an input signal corresponding to the operation and outputs it to the processing circuitry 17. The input interface 30 is one example of an input unit.

The display 40 is constituted by a general display output device such as a liquid crystal display or an organic light emitting diode (OLED) display. The display 40 displays various kinds of information under the control of the processing circuitry 17. The display 40 is one example of a display unit.

FIG. 1 shows the medical image managing apparatus 60 and the medical image processing apparatus 70 which are external devices of the ultrasonic diagnostic apparatus 10. The medical image managing apparatus 60 is, for example, a digital imaging and communications in medicine (DICOM) server, and is connected to a device such as the ultrasonic diagnostic apparatus 10 so that data can be transmitted and received via the network N. The medical image managing apparatus 60 manages a medical image such as an ultrasonic image generated by the ultrasonic diagnostic apparatus 10 as a DICOM file.

The medical image processing apparatus 70 is connected to devices such as the ultrasonic diagnostic apparatus 10 and the medical image managing apparatus 60 so that data is transmitted and received via the network N. An Example of the medical image processing apparatus 70 includes a workstation that performs various image processing on the ultrasonic image generated by the ultrasonic diagnostic apparatus 10 and a portable information processing terminal such as a tablet terminal. It should be noted that the medical image processing apparatus 70 is an offline apparatus and may be an apparatus capable of reading an ultrasonic image generated by the ultrasonic diagnostic apparatus 10 via a portable storage medium.

Subsequently, functions of the ultrasonic diagnostic apparatus 10 will be described.

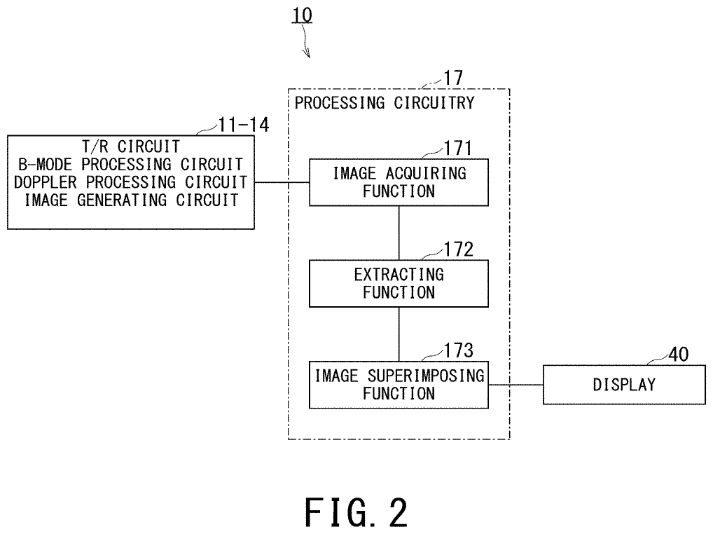

FIG. 2 is a block diagram showing functions of the ultrasonic diagnostic apparatus 10.

The processing circuitry 17 reads out and executes a computer program stored in a non-transitory computer readable medium such as the main memory 18 or a memory directly incorporated in the processing circuitry 17, thereby realizing an image acquiring function 171, an extracting function 172, and an image superimposing function 173. Hereinafter, a case where the functions 171 to 173 function as software will be described as an example. All or a part of the functions 171 to 173 may be provided as a circuit or the like of ASIC etc. in the ultrasonic diagnostic apparatus 10.

The image acquiring function 171 controls the T/R circuit 11, the B-mode processing circuit 12, the Doppler processing circuit 13, the image generating circuit 14, etc., and executes a scan using the ultrasonic probe 20, thereby acquires an ultrasonic image (for example, live images). Specifically, the image acquiring function 171 acquires a CHI (Contrast Harmonic Imaging) image under the CHI, and acquires a vascular structure image, which is an image including the vascular structure, under an image acquisition mode (hereinafter referred to as “vascular acquisition mode”). The vascular acquisition mode is a mode in which a vascular structure, particularly a microvascular structure such as a capillary vessel is capable of capturing. Hereinafter, although the case where the vascular acquisition mode is the mode capable of capturing the microvascular structure will be described, the present invention is not limited to this case. The vascular acquisition mode may be any mode capable of capturing the vascular structure regardless of the blood vessel diameter.

According to the vascular acquisition mode, it is possible to detect blood flow in a micro blood vessel among blood vessels, and to detect boundary information of the microvascular structure by extracting the blood flow in the microvascular.

For example, the image acquiring function 171 acquires the vascular structure image by adopting a technique shown in the present applicant's Japanese Patent (Japanese Patent No. 3724846) as the vascular acquisition mode. By the way, this technique is a technique that suppresses only motion artifacts while leaving clinically useful minute signals by analyzing the characteristics of motion artifacts, and is sometimes called, for example, “Superb Microvascular Imaging (SMI)”. Hereinafter, this technique is described assuming that the vascular structure image is shown in the above Japanese Patent, but the present invention is not limited to this case.

The extracting function 172 includes a function of extracting a vascular structure based on the vascular structure image generated according to the control by the image acquiring function 171. The extraction of the vascular structure by the extracting function 172 may be performed by the same method as the extraction of the vascular structure from the color Doppler image or the power Doppler image.

The image superimposing function 173 includes a function of superimposing data indicating the vascular structure extracted by the extracting function 172 on the contrast image acquired by the image acquiring function 171, thereby generating a superimposed image.

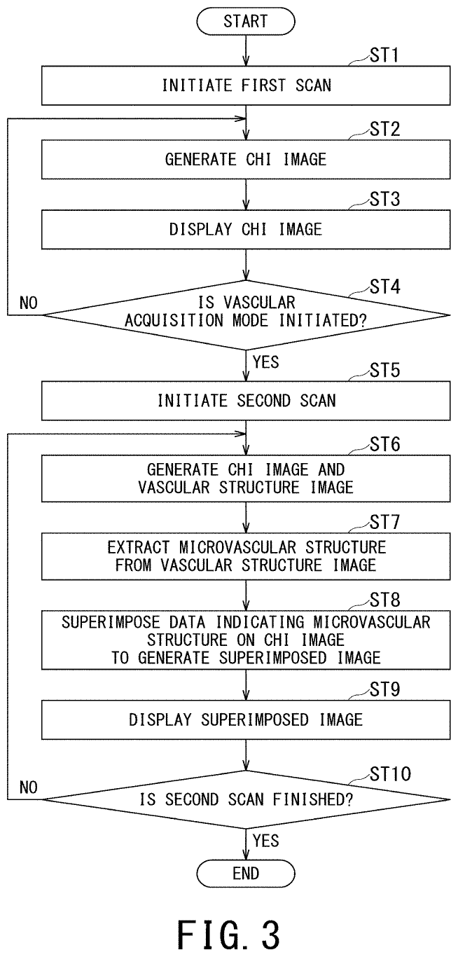

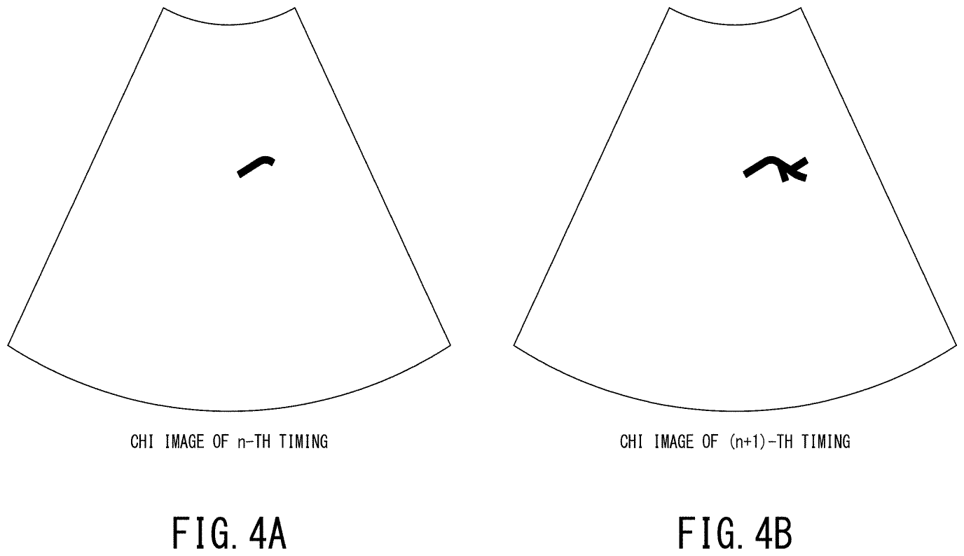

Details of the functions of the functions 171 to 173 will be described later with reference to FIGS. 3 to 10.

Subsequently, an operation of the ultrasonic diagnostic apparatus 10 will be described.

FIG. 3 is a flowchart showing the operation of the ultrasonic diagnostic apparatus 10. In FIG. 3, the reference numerals assigned “ST” with numerals indicate the respective steps of the flowchart.

After the initiate of the injection of the contrast agent, the image acquiring function 171 controls the T/R circuit 11, the B-mode processing circuit 12, the Doppler processing circuit 13, the image generating circuit 14, and the like, and initiates a first scan using the ultrasonic probe 20 (step ST1). By step ST1, the image acquiring function 171 acquires the CHI image under the CHI.

Each of FIGS. 4A and 4B is a diagram showing an example of a CHI image. FIG. 4A shows a CHI image of the n-th timing. FIG. 4B is a diagram showing a CHI image of the (n+1)-th timing. When the time elapses from the timing shown in FIG. 4A to the timing shown in FIG. 4B, the contrast agent advances.

Returning to the description of FIG. 3, the image acquiring function 171 controls the image generating circuit 14 and the like to generate a CHI image (step ST2), and displays the CHI image on the display 40 (step ST3).

The image acquiring function 171 determines whether or not the late stage of contrast has been reached, that is, whether or not to initiate an image superimposing (or vascular acquisition mode) (step ST4). In step ST4, the image acquiring function 171 determines whether the time based on the timing of the initiation of the injection of the contrast agent has elapsed, or the timing of the late stage of contrast has been reached based on image recognition of the CHI image. If it is determined as “NO” in step ST4, that is, if it is determined not to initiate the image superimposing, the image acquiring function 171 generates a CHI image at the next timing (step ST2).

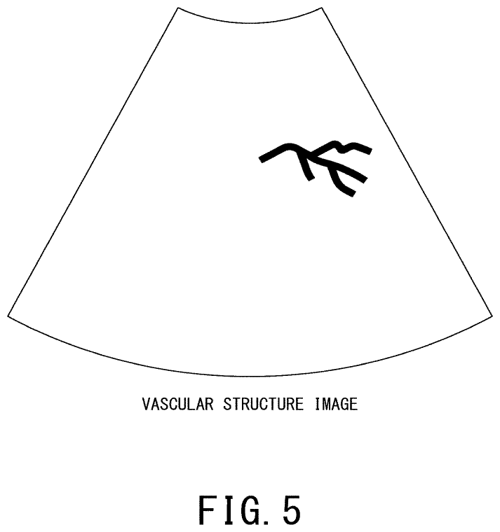

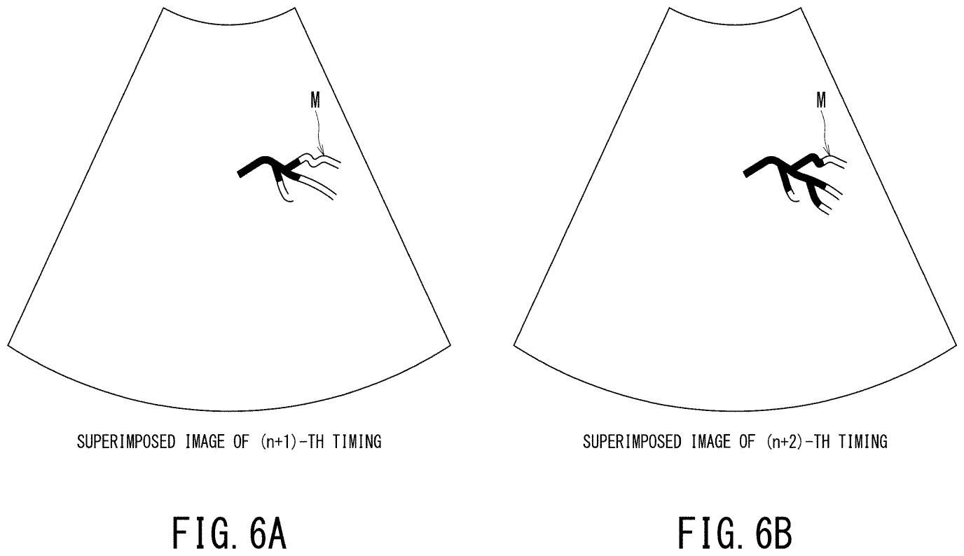

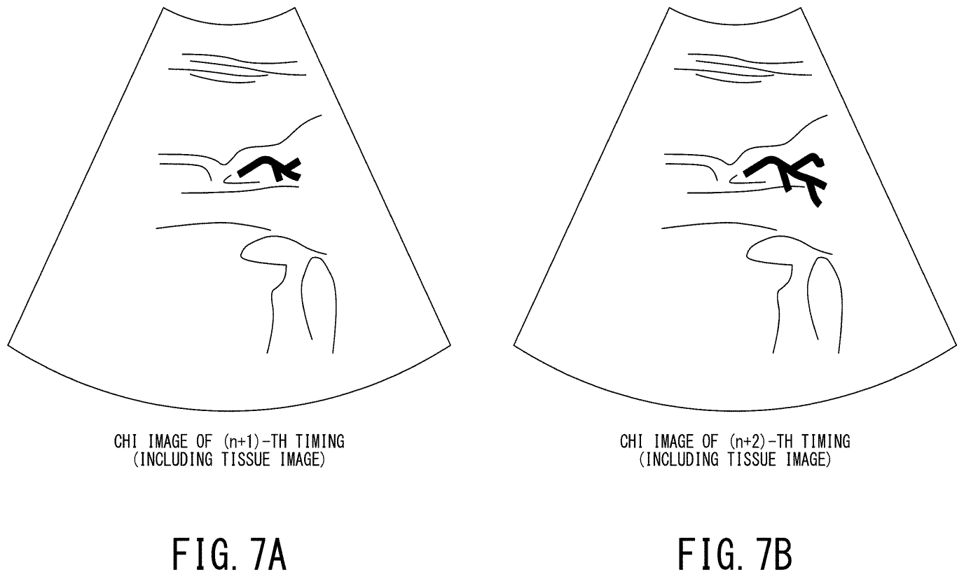

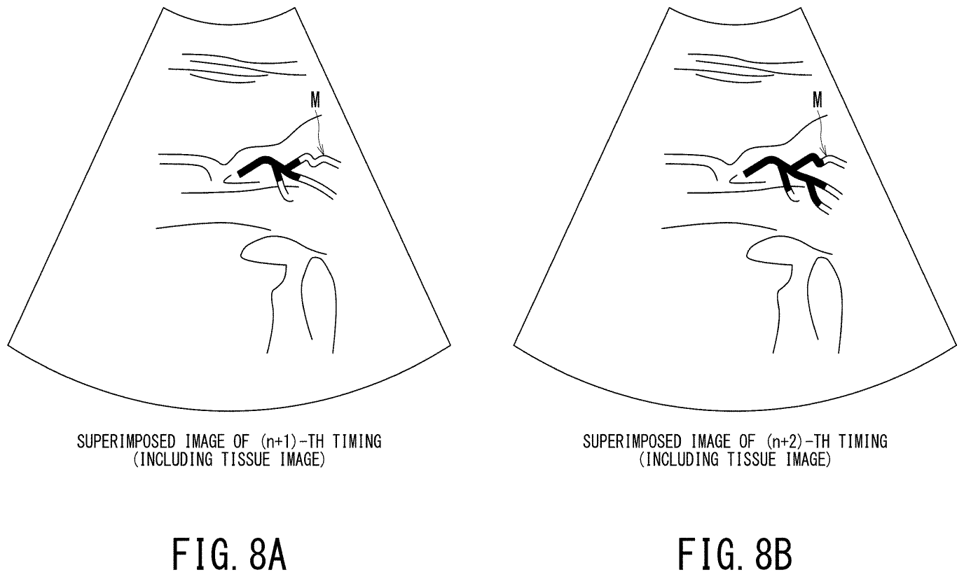

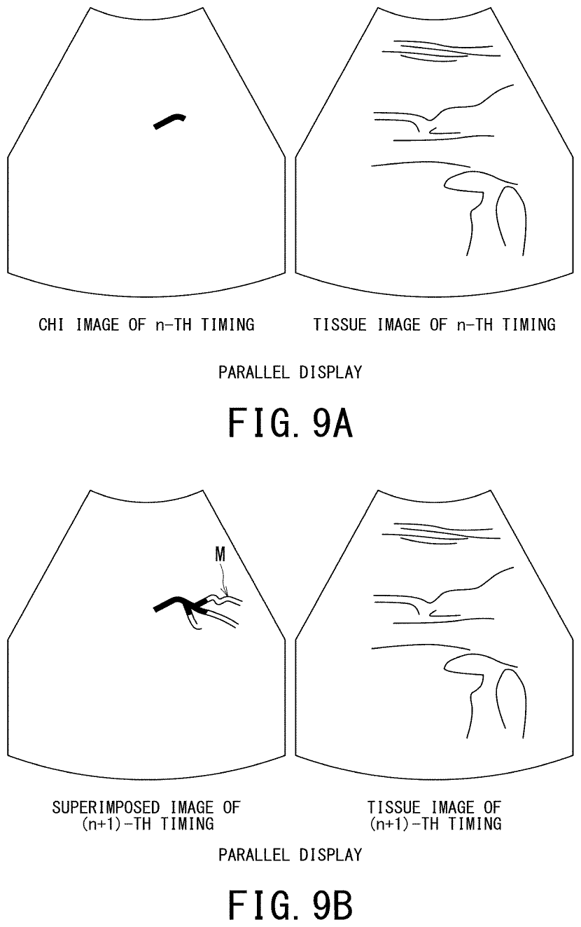

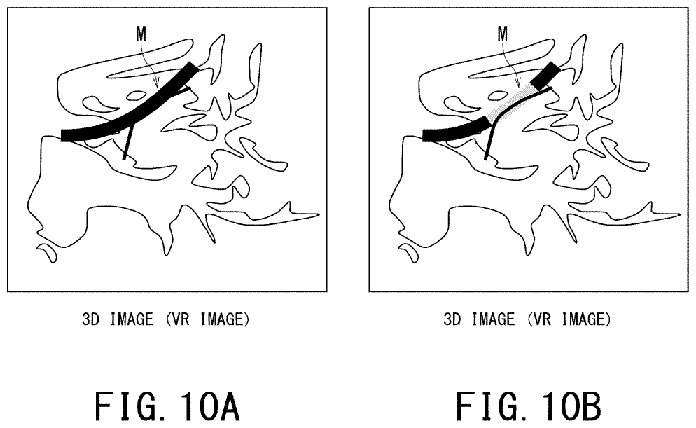

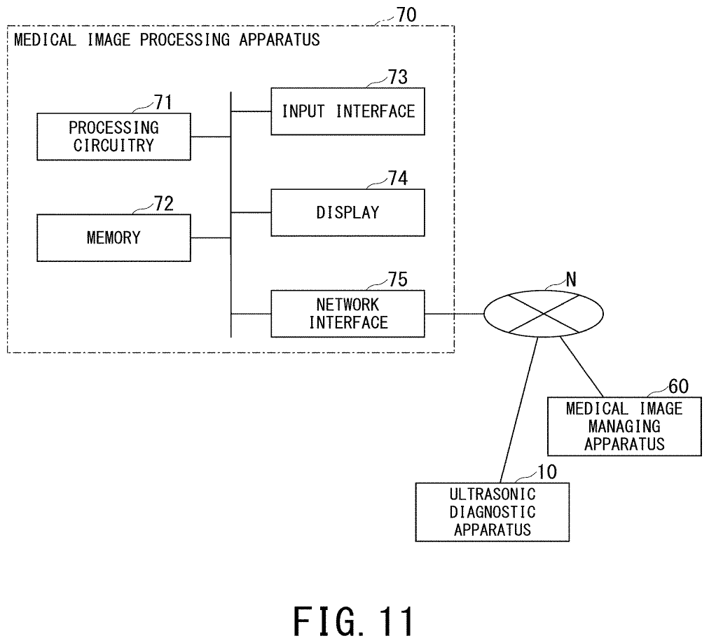

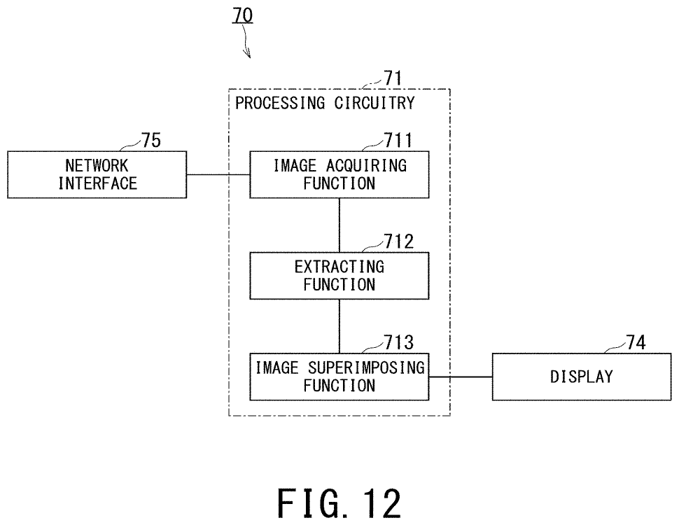

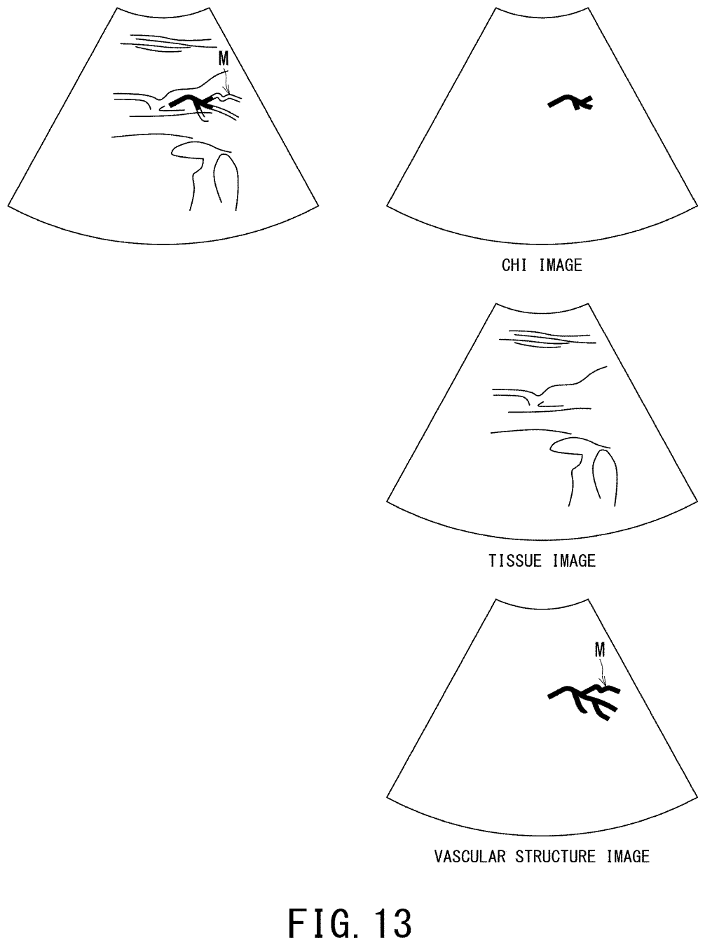

On the other hand, if it is determined as “YES” in step ST4, that is, if it is determined that the image superimposing is initiated, after the initiate of the injection of the contrast agent, the image acquiring function 171 controls the T/R circuit 11, the B-mode processing circuit 12, the Doppler processing circuit 13, the image generating circuit 14 and the like, and initiates a second scan using the ultrasonic probe 20 (step ST5). By step ST5, the image acquiring function 171 acquires the CHI image by CHI, and acquires a vascular structure image by the vascular acquisition mode. FIG. 5 is a diagram showing an example of a vascular structure image showing the microvascular structure.

Returning to the description of FIG. 3, the image acquiring function 171 controls the image generating circuit 14 and the like to generate a CHI image and a vascular structure image (step ST6). The extracting function 172 extracts the microvascular structure based on the vascular structure image generated in step ST6 (step ST7). The extraction of the microvascular structure in step ST7 may be performed by the same method as the extraction of the vascular structure from the color Doppler image or the power Doppler image.

The image superimposing function 173 superimposes data indicating the microvascular structure extracted in step ST7 on the CHI image generated in step ST6 to generate a superimposed image (step ST8). The data indicating the microvascular structure is, for example, the outline of the microvascular structure.

Each of FIGS. 6A and 6B is a diagram showing a first example of the superimposed image. FIG. 6A shows a CHI image of the (n+1)-th timing and an outline M of the microvascular structure superimposed thereon. FIG. 6B shows a CHI image of the (n+2)-th timing and an outline M of the microvascular structure superimposed thereon. When time elapses from the timing shown in FIG. 6A to the timing shown in FIG. 6B, the contrast agent advances.

By generating the superimposed image shown in FIGS. 6A and 6B in which the outline M of the microvascular structure is superimposed, it is possible for the operator to clearly see the microvascular structure, and also visually recognize how the contrast agent flows into the microvascular structure.

Returning to the description of FIG. 3, the image superimposing function 173 displays the superimposed image on the display 40 (step ST9). The image superimposing function 173 determines whether or not to finish the second scan initiated in step ST5 (step ST10). If it is determined as “YES” in step ST10, that is, if it is determined to finish the second scan initiated in step ST5, the ultrasonic diagnostic apparatus 10 finishes the operation.

On the other hand, if it is determined as “NO” in step ST10, that is, if it is determined not to finish the second scan initiated in step ST5, the image acquiring function 171 generates a CHI image and a blood vessel structure image at the next timing (step ST6).

As described with reference to FIG. 3, the ultrasonic diagnostic apparatus 10 performs the processing of a set including steps ST6 to ST9 once. Thereby, the ultrasonic diagnostic apparatus 10 is capable of displaying one superimposed image based on one CHI image on the display 40. In addition, the ultrasonic diagnostic apparatus 10 performs the processing of a set including steps ST6 to ST9 m (“m” is an integer of 2 or more) times. Thereby, the ultrasonic diagnostic apparatus 10 is sequentially capable of displaying m superimposed images based on the m CHI images on the display 40. Specifically, the ultrasonic diagnostic apparatus 10 sequentially generates m CHI images (step ST6), sequentially generates m superimposed images by superimposing the outline M of the vascular structure on each of the CHI images of the m CHI images sequentially generated (steps ST7 and ST8), and sequentially displays m superimposed images generated sequentially on the display 40 (step ST9).

According to the ultrasonic diagnostic apparatus 10, it is possible to provide an image capable of observing a contrast image in the CHI in real time or substantially real time in addition to the microvascular structure in the vascular acquisition mode. Accordingly, it is possible to easily provide an image suitable for diagnosis to the operator without complicated operations.

2. First Modification

The image superimposing function 173 is not limited to the function for superimposing data indicating the microvascular structure on the CHI image. For example, the data indicating the microvascular structure may be further superimposed on an image acquired by superimposing the tissue image on the CHI image.

Each of FIGS. 7A and 7B is a diagram showing a superimposed image according to a comparative example. FIG. 7A shows a CHI image of the (n+1)-th timing and a tissue image superimposed thereon. FIG. 7B shows a superimposed image of the (n+2)-th timing and a tissue image superimposed thereon. When time elapses from the timing shown in FIG. 7A to the timing shown in FIG. 7B, the contrast agent advances.

On the other hand, each of FIGS. 8A and 8B is a diagram showing a second example of the superimposed image according to the present embodiment. FIG. 8A shows a CHI image and a tissue image of the (n+1)-th timing, and an outline M of the microvascular structure superimposed thereon. FIG. 8B shows a CHI image and a tissue image of the (n+2)-th timing, and an outline M of the microvascular structure superimposed thereon. When the time elapses from the timing shown in FIG. 8A to the timing shown in FIG. 8B, the contrast agent advances. Here, the tissue image indicates a B-mode image. The B-mode image includes a so-called B-mode image generated by a scan different from the CHI image, and a fundamental image generated by the same scan as the CHI image generated based on the reflected wave data (reception signal) of the fundamental wave component.

As shown in FIGS. 8A and 8B, the superimposed image in which the outline M of the microvascular structure is superimposed is generated. Thereby, contrary to FIGS. 7A and 7B, it is possible for the operator to clearly see the microvascular structure while confirming the scan position based on the tissue image, and also visually recognize how the contrast agent flows into the microvascular structure.

3. Second Modification

The image acquiring function 171 generates and displays a tissue image and a CHI image by the first scan in step ST1 of FIG. 3, and generates and displays a tissue image, a CHI image, and a vascular structure image by the second scan in step ST5.

Each of FIGS. 9A and 9B is a diagram showing an example of a display image.

As shown in FIG. 9A, in the early stage of contrast, a parallel display including a CHI image and a tissue image is used. Then, when the latter stage of contrast is reached, the image superimposing function 173 switches the parallel display of the CHI image and the tissue image to the parallel display of a superimposed image and a tissue image (shown in FIG. 9B). Thereby, it is possible for the operator to observe the vascular structure without complicated operations.

4. Third Modification

The technical idea of the present invention may also be applied to three-dimensional display and four-dimensional display (space and time). In the latter stage of contrast enhancement, when the data indicating the microvascular structure based on the vascular structure image is superimposed on a three-dimensional image in which a tissue image is superimposed on a CHI image, the data indicating the microvascular structure is displayed translucently.

Each of FIGS. 10A and 10B is a diagram for explaining a superimposed image (three-dimensional image) according to the embodiment. FIG. 10A shows a superimposed image (three-dimensional image) according to a comparative example. FIG. 10B shows a superimposed image (three-dimensional image) according to the third modification.

In FIG. 10A, the operator cannot visually recognize a microvascular structure existing on the back side along the projection direction of the rendering processing, among two outlines M of partially overlapping microvascular structures. On the other hand, in FIG. 10B, the image superimposing function 173 performs a translucent processing on a portion of the microvascular structure appearing on the near side along the direction, among the entire microvascular structure. As a result, according to FIG. 10B, it is possible for the operator to visually recognize the microvascular structure on the back side without performing a rotation operation (changing the line-of-sight direction) of the superimposed image based on a three-dimensional image. According to FIG. 10B, it is possible for the operator to capture the outline M of the microvascular structure in three dimensions.

It should be noted that the present invention is not limited to the case where the translucent processing is performed on a portion of the microvascular structure appearing on the near side along the direction, among the entire microvascular structure. For example, the image superimposing function 173 may perform processing for assigning the part a color different from the other part of the microvascular structure. Accordingly, it is possible to prompt the operator to rotate the superimposed image based on a three-dimensional image (changing the line-of-sight direction).

5. Medical Image Processing Apparatus

FIG. 11 is a schematic diagram showing a configuration of a medical image processing apparatus according to a present embodiment.

FIG. 11 shows a medical image processing apparatus 70 according to a present embodiment. The medical image processing apparatus 70 is a medical image managing apparatus (image server), a workstation, a medical interpretation terminal, or the like, and is provided on a medical image system connected via a network. The medical image processing apparatus 70 may be an offline apparatus.

The medical image processing apparatus 70 includes processing circuitry 71, a memory 72, an input interface 73, a display 74, and a network interface 75. The processing circuitry 71, the memory 72, the input interface 73, and the display 74 have the same configuration as the processing circuitry 17, the main memory 18, the input interface 30, and the display 40 shown in FIGS. 1 and 2, respectively. Therefore, those explanations are omitted.

The network interface 75 includes a connector that conforms to a parallel connection specification or a serial connection specification. When the medical image processing apparatus 70 is provided on the medical image system, the network interface 75 transmits/receives information to/from an external apparatus on the network. For example, the network interface 75 receives medical image data such as CT image data from an external device under the control of the processing circuitry 71.

Subsequently, functions of the medical image processing apparatus 70 will be described.

FIG. 12 is a block diagram showing functions of the medical image processing apparatus 70.

The processing circuitry 71 reads out and executes a computer program stored in a non-transitory computer readable medium such as the memory 72 or a memory directly incorporated in the processing circuitry 71, thereby realizing an image acquiring function 711, an extracting function 712, and an image superimposing function 713. Hereinafter, a case where the functions 711 to 713 are realized by executing the computer program of the medical image processing apparatus 70 will be described as an example. All or part of the functions 711 to 713 may be provided in the medical image processing apparatus 70 as a circuit such as an ASIC.

The image acquiring function 711 includes a function of acquiring the corresponding CHI image, tissue image, and vascular structure image from the medical image managing apparatus 60 or the ultrasonic diagnostic apparatus 10 via the network interface 75. The image acquiring function 711 is one example of an image acquiring unit.

The extracting function 712 includes a function equivalent to the extracting function 172 shown in FIG. 2. The extracting function 712 is one example of an extracting unit.

The image superimposing function 713 includes a function equivalent to the image superimposing function 173 shown in FIG. 2. The image superimposing function 713 is one example of an image superimposing unit.

An operation of the medical image processing apparatus 70 is the same as that of the ultrasonic diagnostic apparatus 10 shown in FIG. 3.

According to the medical image processing apparatus 70, it is possible to provide an image capable of observing a contrast image in the CHI in addition to the microvascular structure in the vascular acquisition mode. Accordingly, it is possible to easily provide an image suitable for diagnosis to the operator without complicated operations.

6. Fourth Modification

As described above, the case where the ultrasonic diagnostic apparatus 10 displays the CHI image, the tissue image, and the data indicating the microvascular tissue superimposed on each other during scanning has been described. However, when the image is recognized again after the examination using the medical image processing apparatus 70, the image superimposing function 713 may switch the display of the superimposed image to a parallel display in accordance with an operation using the input interface 73. The parallel display may be a parallel display of the CHI image and the vascular structure image as elements of the superimposed image, or a parallel display of the CHI image, the vascular structure image and the tissue image.

FIG. 13 is a diagram for explaining a concept of switching the display of the superimposed image to the parallel display of the CHI image, the vascular structure image and the tissue image as elements thereof.

The left side of FIG. 13 shows a single display example of the superimposed image. The right side of FIG. 13 shows a parallel display example of a CHI image, a vascular structure image, and a tissue image. By switching the single display on the left side to the parallel display on the right side, it is possible to perform detailed observation and measurement for each image type. Further, by saving again after the parallel display, it is possible to reduce and save a desired amount of data.

According to at least one of the embodiments described above, it is possible to provide an image capable of observing a contrast image in addition to a vascular structure, particularly a microvascular structure such as a capillary vessel.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
processing circuitry configured to
acquire a contrast image and a vascular structure image which includes the vascular structure in an image acquisition mode capable of capturing the vascular structure,
extract data indicating the vascular structure from the vascular structure image,
superimpose the data indicating the vascular structure on the contrast image to generate a superimposed image, and
display the superimposed image on a display.

2. The ultrasonic diagnostic apparatus according to claim 1, wherein
the data indicating the vascular structure is an outline of the vascular structure.

3. The ultrasonic diagnostic apparatus according to claim 1, wherein
the processing circuitry is configured to acquire the vascular structure image according to a mode capable of capturing a microvascular structure as the image acquisition mode.

4. The ultrasonic diagnostic apparatus according to claim 2, wherein
the processing circuitry is configured to acquire the vascular structure image according to a mode capable of capturing a microvascular structure as the image acquisition mode.

5. The ultrasonic diagnostic apparatus according to claim 1, wherein
the processing circuitry is configured to superimpose the data indicating the vascular structure on an image in which a tissue image is superimposed on the contrast image to generate the superimposed image.

6. The ultrasonic diagnostic apparatus according to claim 2, wherein
the processing circuitry is configured to superimpose the outline of the vascular structure on an image in which a tissue image is superimposed on the contrast image to generate the superimposed image.

7. The ultrasonic diagnostic apparatus according to claim 1, wherein
the processing circuitry is configured to switch a parallel display of the contrast image and a tissue image on the display to a parallel display of the superimposed image and the tissue image on the display.

8. The ultrasonic diagnostic apparatus according to claim 2, wherein
the processing circuitry is configured to switch a parallel display of the contrast image and a tissue image on the display to a parallel display of the superimposed image and the tissue image on the display.

9. The ultrasonic diagnostic apparatus according to claim 1, wherein
the contrast image comprises multiple contrast images, and
the processing circuitry configured to
sequentially acquire multiple contrast images,
sequentially generate multiple superimposed images by superimposing the data indicating the vascular structure on each contrast image of the multiple contrast images, and
sequentially display the multiple superimposed images on the display.

10. The ultrasonic diagnostic apparatus according to claim 2, wherein
the contrast image comprises multiple contrast images, and
the processing circuitry configured to
sequentially acquire multiple contrast images,
sequentially generate multiple superimposed images by superimposing the outline of the vascular structure on each contrast image of the multiple contrast images, and
sequentially display the multiple superimposed images on the display.

11. The ultrasonic diagnostic apparatus according to claim 1, wherein
the processing circuitry is configured to perform a translucent processing on a portion of the vascular structure appearing on a near side along a projection direction when rendering the superimposed image, the superimposed image being a three-dimensional image.

12. The ultrasonic diagnostic apparatus according to claim 2, wherein
the processing circuitry is configured to perform a translucent processing on a portion of the vascular structure appearing on a near side along a projection direction when rendering the superimposed image, the superimposed image being a three-dimensional image.

13. The ultrasonic diagnostic apparatus according to claim 1, wherein
the processing circuitry is configured to perform processing of assigning a part a color different from the other part of the vascular structure, the part being present on a front side along a projection direction when rendering the superimposed image, and the superimposed image being a three-dimensional image.

14. The ultrasonic diagnostic apparatus according to claim 2, wherein
the processing circuitry is configured to perform processing of assigning a part a color different from the other part of the vascular structure, the part being present on a front side along a projection direction when rendering the superimposed image, and the superimposed image being a three-dimensional image.

15. The ultrasonic diagnostic apparatus according to claim 1, wherein
the processing circuitry is configured to analyze a feature of a motion artifact to acquire the vascular structure image by a method of suppressing only the motion artifact while leaving a predetermined minute signal.

16. The ultrasonic diagnostic apparatus according to claim 2, wherein
the processing circuitry is configured to analyze a feature of a motion artifact to acquire the vascular structure image by a method of suppressing only the motion artifact while leaving a predetermined minute signal.

17. A medical image processing apparatus comprising:
processing circuitry configured to
acquire a contrast image and a vascular structure image which includes the vascular structure in an image acquisition mode capable of capturing the vascular structure,
extract data indicating the vascular structure from the vascular structure image,
superimpose the data indicating the vascular structure on the contrast image to generate a superimposed image, and
display the superimposed image on a display.

18. The medical image processing apparatus according to claim 17, wherein
the processing circuitry is configured to switch a display of the superimposed image to a parallel display of the contrast image as an element of the superimposed image and the vascular structure image, or to a parallel display of the contrast image as an element of the superimposed image, the vascular structure image and the tissue image.

19. A non-transitory computer readable medium comprising:
functions of
acquiring a contrast image and a vascular structure image which includes the vascular structure in an image acquisition mode capable of capturing the vascular structure,
extracting data indicating the vascular structure from the vascular structure image,
superimposing the data indicating the vascular structure on the contrast image to generate a superimposed image, and
displaying the superimposed image on a display.
